(19) United States
(12) Patent Application Publication
(10) Pub. No.: US 2018/0035913 A1
(43) Pub. Date: Feb. 8, 2018

(54) POSITION DETECTION SYSTEM AND OPERATION METHOD OF POSITION DETECTION SYSTEM

(71) Applicant: OLYMPUS CORPORATION, Tokyo (JP)

(72) Inventors: Atsushi CHIBA, Tokyo (JP); Yusuke SUZUKI, Tokyo (JP)

(73) Assignee: OLYMPUS CORPORATION, Tokyo (JP)

(21) Appl. No.: 15/788,095

(22) Filed: Oct. 19, 2017

Related U.S. Application Data
(63) Continuation of application No. PCT/JP2016/081695, filed on Oct. 26, 2016.

(30) Foreign Application Priority Data
Dec. 2, 2015 (JP) 2015-236127

(51) Int. Cl. A61B 5/06; A61B 5/00; A61B 1/04; A61B 1/00

(52) U.S. Cl. CPC A61B 5/062; A61B 1/00158; A61B 5/742; A61B 1/041; A61B 1/00032

(57) ABSTRACT

A position detection system includes: a detection target including a magnetic field generator and a permanent magnet; detection coils; a guidance magnetic field generator including a magnetic field generation source configured to generate a guidance magnetic field for guiding the detection target, and a driving mechanism configured to change at least one of a position and a posture of the magnetic field generation source, wherein at least a part of the guidance magnetic field generator is formed of a conductor that generates an interference magnetic field; a guidance magnetic field controller configured to control an operation of the driving mechanism; and a position detector configured to calculate at least one of a position and a posture of the detection target by using: detection signals respectively output from the detection coils; and at least one of a position and a posture of the conductor.

### CROSS-REFERENCE TO RELATED APPLICATION

This application is a continuation of PCT International Application No. PCT/JP2016/081695 filed on Oct. 26, 2016 which claims the benefit of priority from Japanese Patent Application No. 2015-236127 filed on Dec. 2, 2015, the entire contents of which are incorporated herein by reference.

### BACKGROUND

The present disclosure relates to a position detection system and an operation method of the position detection system.

In recent years, a capsule medical apparatus introduced into a subject to acquire various types of information about the subject or to administer a drug to the subject has been developed. As an example, a capsule endoscope formed in a size which may be introduced into the gastrointestinal tract of a subject is known. The capsule endoscope has an imaging function and a wireless communication function inside a capsule-shaped casing, and after being swallowed into the subject, the capsule endoscope performs imaging while moving inside the gastrointestinal tract, and wirelessly transmits sequentially image data of images of the inside of an organ of the subject.

A system for performing position detection using such a capsule medical apparatus as a detection target has also been developed. For example, JP 2008-132047 A discloses a position detection system which includes a capsule medical apparatus having therein a magnetic field generating coil which generates a magnetic field for position detection by receiving power supply, and detection coils which detect the magnetic field generated by the magnetic field generating coil outside a subject, and performs calculation for detecting a position of the capsule medical apparatus based on the strength of the magnetic field detected by the detection coils.

In addition, a system for guiding a capsule medical apparatus introduced into a subject by a magnetic field has been proposed. For example, JP 2006-68501 A discloses a magnetic guidance medical system for guiding a capsule medical apparatus by introducing a capsule medical apparatus having therein a permanent magnet into a subject, providing a magnetic field generation unit outside the subject, and causing the magnetic field generation unit to move so as to change the magnetic field acting on the permanent magnet in the capsule medical apparatus.

### SUMMARY

A position detection system according to one aspect of the present disclosure may include: a detection target including a magnetic field generator configured to generate an alternating magnetic field for position detection and a permanent magnet provided therein, the detection target being adapted to be introduced into a subject; a plurality of detection coils arranged outside the subject, each of the detection coils detecting the alternating magnetic field and outputting a detection signal; a guidance magnetic field generator including a magnetic field generation source configured to generate a guidance magnetic field for guiding the detection target, and a driving mechanism configured to change at least one of a position and a posture of the magnetic field generation source, wherein at least a part of the guidance magnetic field generator is formed of a conductor that generates an interference magnetic field by an action of the alternating magnetic field; a guidance magnetic field controller configured to control an operation of the driving mechanism; and a position detector configured to calculate at least one of a position and a posture of the detection target by using: a plurality of the detection signals respectively output from the detection coils; and at least one of a position and a posture of the conductor.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a schematic diagram illustrating an outline of a position detection system according to a first embodiment of the present disclosure;

FIG. 2 is a schematic diagram illustrating an example of an internal structure of a capsule endoscope illustrated in FIG. 1;

FIG. 3 is a diagram illustrating a detailed configuration of the position detection system illustrated in FIG. 1;

FIG. 4 is a schematic view illustrating a configuration example of a guidance magnetic field generating device illustrated in FIG. 3;

FIG. 5 is a block diagram illustrating a configuration example of a magnet drive unit illustrated in FIG. 4;

FIG. 6 is a flowchart illustrating a position detection method according to the first embodiment of the present disclosure;

FIG. 7 is a schematic view illustrating an example of a positional relationship among an external permanent magnet, a capsule endoscope, and a plurality of detection coils;

FIG. 8 is a schematic view illustrating an example of a positional relationship among an external permanent magnet, the capsule endoscope, and the plurality of detection coils;

FIG. 9 is a table illustrating an example of correction coefficients for calculating a correction value with a coordinate in the vertical direction of the external permanent magnet as an input value;

FIG. 10 is a set of graphs illustrating a relationship between the coordinate in the vertical direction of the external permanent magnet and coordinates in respective directions of the capsule endoscope before and after correction;

FIG. 11 is a schematic view illustrating a partial configuration of a position detection system according to a third embodiment of the present disclosure; and

FIG. 12 is a schematic view illustrating a partial configuration of a position detection system according to a fourth embodiment of the present disclosure.

### DETAILED DESCRIPTION

Hereinafter, a position detection system and a position detection method according to embodiments of the present disclosure will be described with reference to the drawings. In the embodiments to be described below, as one form of a detection target of which a position and a posture are objects to be detected by the position detection system, a capsule endoscope is exemplified which is orally introduced into a subject and captures images of the inside of the gastrointestinal tract of the subject. However, the present disclosure is not limited by these embodiments. That is, the present disclosure may be applied to detection of positions and postures of various devices introduced into a subject, for example, a capsule endoscope which moves inside a lumen from the esophagus to the anus of the subject, a capsule medical apparatus which delivers a medicine or the like into the subject, a capsule medical apparatus which includes a pH sensor for measuring a pH in the subject.

In the following description, each figure only schematically illustrates a shape, a size, and a positional relationship to the extent that the contents of the present disclosure may be understood. Therefore, the present disclosure is not limited exclusively to the shape, the size, and the positional relationship exemplified in each figure. In the drawings, the same parts are denoted by the same reference signs.

### First Embodiment

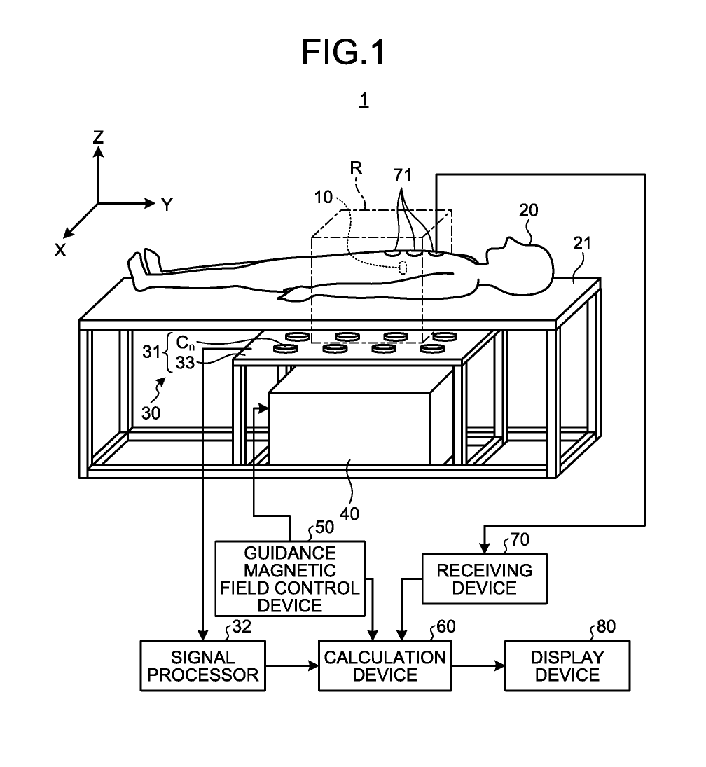

FIG. 1 is a schematic diagram illustrating an outline of a position detection system according to a first embodiment of the present disclosure. As illustrated in FIG. 1, a position detection system 1 according to the first embodiment is a system which detects a position of a capsule endoscope introduced into a subject 20 to capture an image of the inside of the subject 20, as an example of a detection target. The position detection system 1 includes a capsule endoscope 10, a bed 21, a magnetic field detection device 30, a guidance magnetic field generating device 40, a guidance magnetic field control device 50, a calculation device 60 (position detection calculation device), a receiving device 70, and a display device 80. On the bed 21, the subject 20 is placed. The magnetic field detection device 30 detects a position-detecting magnetic field generated by the capsule endoscope 10. The guidance magnetic field generating device 40 generates a magnetic field for guiding the capsule endoscope 10. The guidance magnetic field control device 50 controls an operation of the guidance magnetic field generating device 40. The calculation device 60 (position detection calculation device) performs a calculation process for detection of a position of the capsule endoscope 10 and the like based on a detection signal of the position-detecting magnetic field output from the magnetic field detection device 30. The receiving device 70 receives a signal wirelessly transmitted from the capsule endoscope 10 via a receiving antenna 71 affixed to the body surface of the subject 20. The display device 80 displays an image output from the calculation device 60 and positional information of the capsule endoscope 10, and the like.

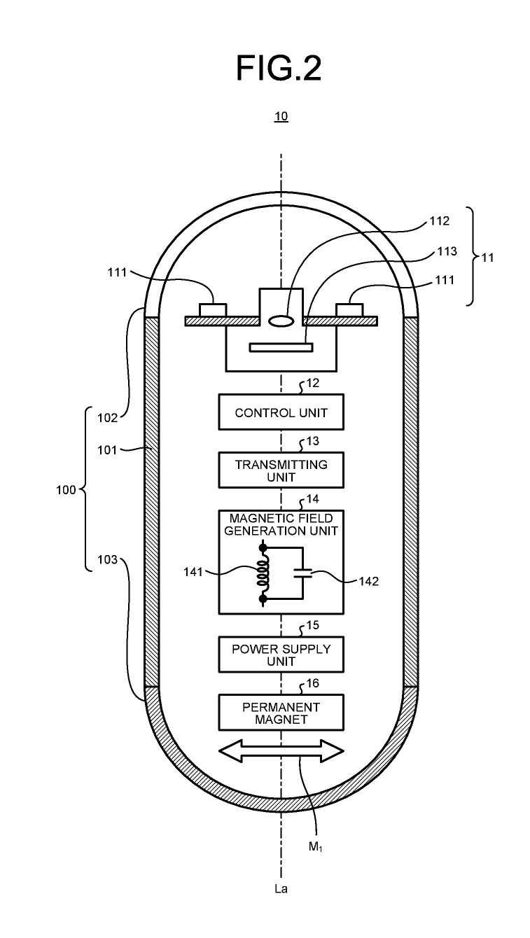

FIG. 2 is a schematic diagram illustrating an example of an internal structure of the capsule endoscope 10 illustrated in FIG. 1. As illustrated in FIG. 2, the capsule endoscope 10 includes a casing 100, an imaging unit 11, a control unit 12, a transmitting unit 13, a magnetic field generation unit 14, a power supply unit 15, and a permanent magnet 16. The casing 100 is capsule-shaped and formed in a size easy to introduce into the subject 20. The imaging unit 11 is accommodated in the casing 100 and captures an image of the inside of the subject 20 to acquire an imaging signal. The control unit 12 controls an operation of each unit of the capsule endoscope 10 including the imaging unit 11, and performs a predetermined signal process to the imaging signal acquired by the imaging unit 11. The transmitting unit 13 wirelessly transmits the imaging signal which has been subjected to the signal process. The magnetic field generation unit 14 generates an alternating magnetic field as a position-detecting magnetic field of the capsule endoscope 10. The power supply unit 15 supplies power to each unit of the capsule endoscope 10.

The casing 100 is an outer casing formed in a size which may be introduced into an organ of the subject 20. The casing 100 has a cylindrical casing 101 having a cylindrical shape and two dome-shaped casings 102 and 103 having a dome shape and respectively closing open ends on both sides of the cylindrical casing 101. The cylindrical casing 101 is formed of a colored member which is substantially opaque to visible light. The dome-shaped casing 102 provided on a side of the imaging unit 11 is formed of an optical member which is transparent to light of a predetermined wavelength band such as visible light. The casing 100 includes the imaging unit 11, the control unit 12, the transmitting unit 13, the magnetic field generation unit 14, the power supply unit 15, and the permanent magnet 16 liquid-tightly. In FIG. 2, the imaging unit 11 is provided on a side of the dome-shaped casing 102 only, but the imaging unit 11 may be further provided on a side of the dome-shaped casing 103. In that case, the dome-shaped casing 103 is also formed of a transparent optical member.

The imaging unit 11 includes illumination units 111, an optical system 112, and an imaging element 113. Each illumination unit 111 has a light source such as an LED, and emits illumination light having a predetermined color component (for example, white light) in a region including an imaging view field of the imaging element 113 to illuminate the inside of the subject 20 through the dome-shaped casing 102. The optical system 112 has one or a plurality of lenses, and condenses light from the subject 20 onto a light-receiving surface of the imaging element 113 to form an image. The imaging element 113 has an image sensor such as a CMOS or a CCD, converts the light received on the light-receiving surface into an electrical signal, and outputs the electrical signal as an imaging signal.

The control unit 12 operates the imaging unit 11 at a predetermined imaging cycle and causes each illumination unit 111 to emit light in synchronization with the imaging cycle. In addition, the control unit 12 performs a predetermined signal process including A/D conversion on the imaging signal generated by the imaging unit 11 to generate image data.

The transmitting unit 13 includes a transmitting antenna. The transmitting unit 13 sequentially acquires the image data subjected to the signal process by the control unit 12 and related information to perform a modulation process, and wirelessly transmits sequentially the modulated signal to the outside via the transmitting antenna.

The magnetic field generation unit 14 includes a magnetic field generating coil 141 which generates a magnetic field by a flow of a current and a capacitor 142 which is connected in parallel with the magnetic field generating coil 141 and forms a resonance circuit together with the magnetic field generating coil 141. The magnetic field generation unit 14 receives power supply from the power supply unit 15 and generates an alternating magnetic field of a predetermined frequency as a position-detecting magnetic field.

The power supply unit 15 includes a power storage unit such as a button battery or a capacitor, and a switch unit such as a magnetic switch or an optical switch. When the power supply unit 15 is configured to have a magnetic switch, switching between ON and OFF states of power is performed by a magnetic field applied from the outside, and in the ON state, the power of the power storage unit is appropriately supplied to each component (the imaging unit 11, the control unit 12, and the transmitting unit 13) of the capsule endoscope 10, and in the OFF state, the supply is stopped.

The permanent magnet 16 is provided to enable the capsule endoscope 10 to be guided by a magnetic field applied from the outside. The permanent magnet 16 is fixedly disposed inside the casing 100 so that a magnetization direction intersects a major axis La of the casing 100. In the case illustrated in FIG. 2, the magnetization direction (an arrow M₁ in FIG. 2) of the permanent magnet 16 is orthogonal to the major axis La.

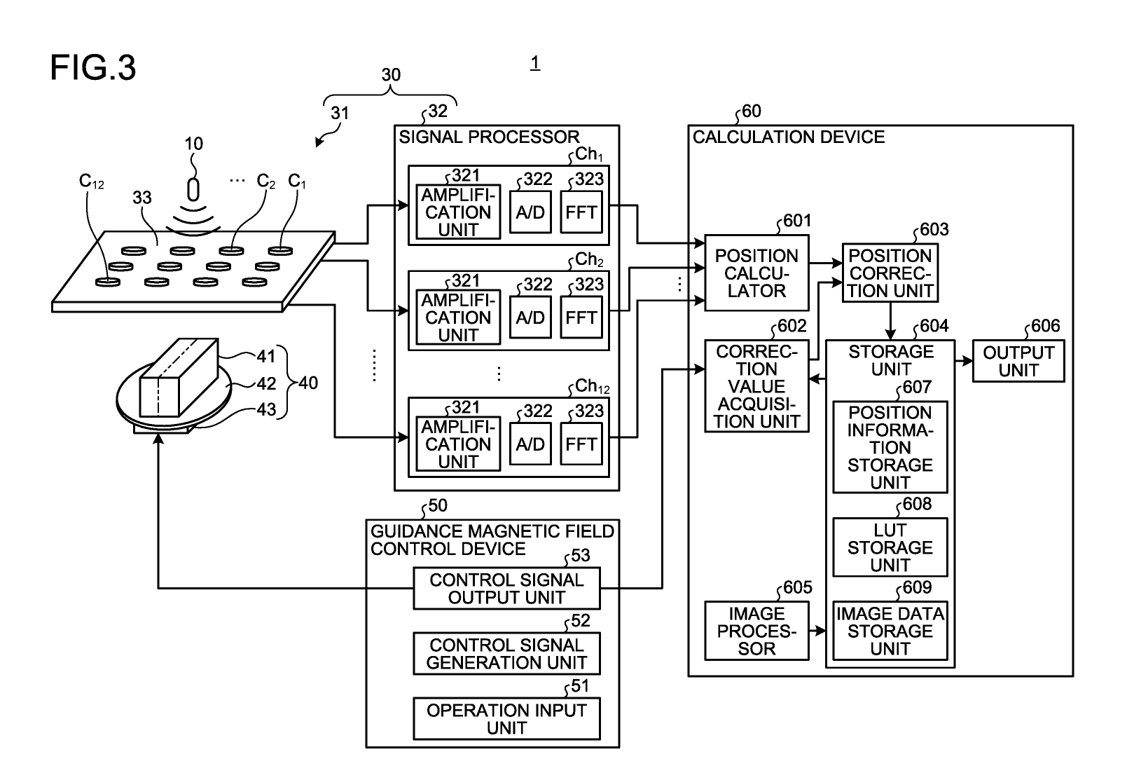

FIG. 3 is a diagram illustrating a detailed configuration of the position detection system 1 illustrated in FIG. 1. The magnetic field detection device 30 illustrated in FIG. 3 includes a coil unit 31, and a signal processor 32. In the coil unit 31, a plurality of detection coils C₁ to C₁₂ is arranged. The signal processor 32 processes detection signals respectively output from the detection coils C₁ to C₁₂.

The detection coils Cₙ (n=1 to 12) are each obtained by winding a wire in a spiral shape, and its size is, for example, about 30 to 40 mm in opening diameter and about 5 mm in height. The detection coils Cₙ are arranged on a main surface of a flat panel 33 formed of a nonmetallic material such as resin. In each of the detection coils Cₙ, a current corresponding to a change in the magnetic field at an arrangement position thereof is generated and output to the signal processor 32. In this sense, the current generated in each of the detection coils Cₙ is nothing less than the detection signal.

The arrangement position and the number of the detection coils in the coil unit 31 are determined depending on a detection target region when detecting the capsule endoscope 10 in the subject 20 to be examined on the bed 21. The detection target region is set in advance depending on conditions such as a movable range of the capsule endoscope 10 and the strength of the position-detecting magnetic field generated by the capsule endoscope 10 in the subject 20 examined on the bed 21. For example, in the case illustrated in FIG. 1, a detection target region R is set as a three-dimensional region including a part of a region above the bed 21.

The signal processor 32 includes a plurality of signal processing channels Ch₁ to Ch₁₂, the signal processing channels Ch₁ to Ch₁₂ corresponding to the detection coils C₁ to C₁₂, respectively. The signal processing channels Chₙ each include an amplification unit 321, an A/D converter (A/D) 322, and an FFT processor (FFT) 323. The amplification unit 321 amplifies a detection signal output from each of the detection coils Cₙ. The A/D converter (A/D) 322 digitally converts the amplified detection signal. The FFT processor (FFT) 323 performs a fast Fourier transform process on the digitally converted detection signal and outputs the detection signal to the calculation device 60.

The guidance magnetic field generating device 40 is disposed on an opposite side of the detection target region R for the capsule endoscope 10 with respect to the coil unit 31, that is, on a lower region side of the coil unit 31, and generates a guidance magnetic field for changing at least one of a position and a posture of the capsule endoscope 10 which has been introduced into the subject 20 on the bed 21. Here, the posture of the capsule endoscope 10 is represented by an elevation angle which is an angle with respect to a horizontal plane of the major axis La (see FIG. 2) of the capsule endoscope 10 with respect to the horizontal plane (XY plane) and a traverse angle (azimuth) of the major axis La rotating about an axis in a vertical direction (Z direction) from a predetermined reference position.

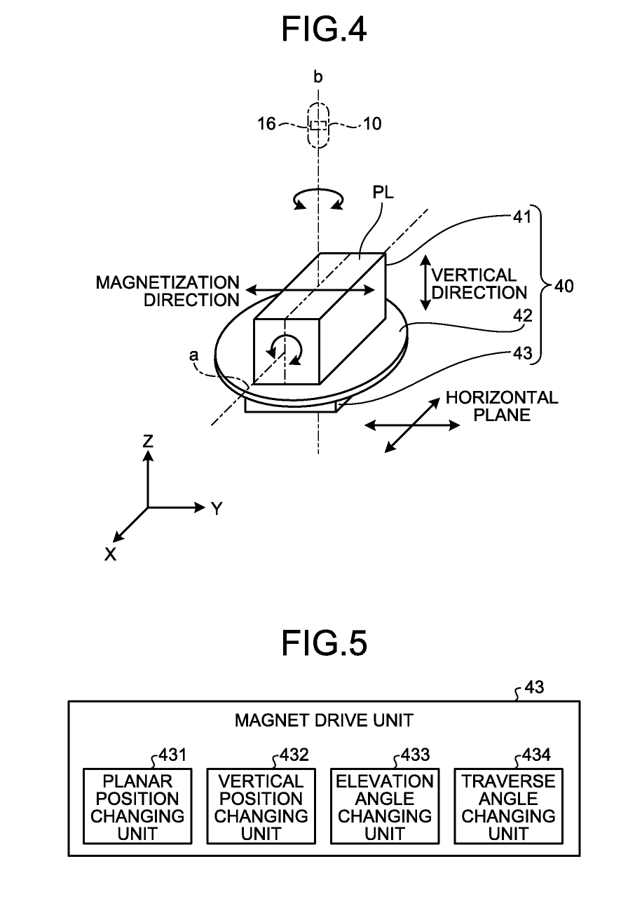

FIG. 4 is a schematic view illustrating a configuration example of the guidance magnetic field generating device 40. As illustrated in FIG. 4, the guidance magnetic field generating device 40 includes a permanent magnet (hereinafter referred to as an external permanent magnet) 41, a support member 42, and a magnet drive unit 43. The external permanent magnet 41 serves as a magnetic field generation source which generates the guidance magnetic field for the capsule endoscope 10. The support member 42 supports the external permanent magnet 41. The magnet drive unit 43 changes at least one of a position and a posture of the external permanent magnet 41 via the support member 42.

The guidance magnetic field generating device 40 is at least partially formed of a conductor. Generally, there exists the position-detecting magnetic field generated by the capsule endoscope 10 in a region where the guidance magnetic field generating device 40 is disposed. Consequently, by the position-detecting magnetic field changing with time, an eddy current flows through the conductor included in the guidance magnetic field generating device 40 and a new magnetic field (interference magnetic field) is generated. Therefore, the conductor included in the guidance magnetic field generating device 40 is a generation source of the interference magnetic field with respect to the position-detecting magnetic field. Since the conductor included in the guidance magnetic field generating device 40 moves and rotates under the control of the guidance magnetic field control device 50, the interference magnetic field also changes with time.

The external permanent magnet 41 is achieved by a bar magnet having a rectangular parallelepiped shape, for example. In that case, in an initial state, the external permanent magnet 41 is disposed so that one plane PL of four planes parallel to a magnetization direction thereof is parallel to the horizontal plane (see FIG. 4). Although the material of the external permanent magnet 41 is not particularly limited, for example, a metal magnet such as a neodymium magnet may be used. When a metal magnet is used as the external permanent magnet 41, the external permanent magnet 41 itself is a generation source of the interference magnetic field. Since the guidance magnetic field generated by the external permanent magnet 41 is stationary, the guidance magnetic field may be separated from the position-detecting magnetic field which is an alternating magnetic field.

The material of the support member 42 is not particularly limited, but when the support member 42 is formed of a conductor such as metal, the support member 42 may also be a generation source of the interference magnetic field.

The magnet drive unit 43 is a driving mechanism which changes a position and a posture of the external permanent magnet 41 via the support member 42. The magnet drive unit 43 includes a motor or the like which translates or rotates the external permanent magnet 41. Since a metal member is used in a commonly used motor, the magnet drive unit 43 may also be a generation source of the interference magnetic field with respect to the position-detecting magnetic field. When the support member 42 is formed of metal and the magnet drive unit 43 is covered by the support member 42 as seen from all the detection coils C₁ to C₁₂ as illustrated in FIG. 3, there is no need to consider the magnet drive unit 43 as a generation source of the interference magnetic field.

FIG. 5 is a block diagram illustrating a configuration example of the magnet drive unit 43. The magnet drive unit 43 includes a planar position changing unit 431, a vertical position changing unit 432, an elevation angle changing unit 433, and a traverse angle changing unit 434. The planar position changing unit 431 translates the external permanent magnet 41 in the horizontal plane. The vertical position changing unit 432 translates the external permanent magnet 41 in the vertical direction. The elevation angle changing unit 433 changes the elevation angle of the external permanent magnet 41 by rotating the external permanent magnet 41 about an axis which passes a center of the external permanent magnet 41, is orthogonal to the magnetization direction of the external permanent magnet 41 and is parallel to the horizontal plane. The traverse angle changing unit 434 changes the traverse angle of the external permanent magnet 41 by rotating the external permanent magnet 41 with respect to an axis in the vertical direction which passes the center of the external permanent magnet 41. Hereinafter, a rotation axis (an axis a illustrated in FIG. 4) used when the elevation angle changing unit 433 changes the elevation angle of the external permanent magnet 41 is referred to as a central axis a, and a rotation axis (an axis b illustrated in FIG. 4) used when the traverse angle changing unit 434 changes the traverse angle of the external permanent magnet 41 is referred to as a vertical axis b.

Through the operation of the magnet drive unit 43 described above, the external permanent magnet 41 and the support member 42 have five degrees of freedom: translation in a three-dimensional space, rotation about the central axis a, and rotation about the vertical axis b.

The guidance magnetic field control device 50 controls the guidance magnetic field generating device 40 in order to achieve guidance desired by a user with respect to the capsule endoscope 10. As illustrated in FIG. 3, the guidance magnetic field control device 50 includes an operation input unit 51, a control signal generation unit 52, and a control signal output unit 53. The operation input unit 51 is used by the user when guiding the capsule endoscope 10 introduced into the subject 20. The control signal generation unit 52 generates a control signal for the magnet drive unit 43 (driving mechanism) based on the operation to the operation input unit 51. The control signal output unit 53 outputs the control signal to the magnet drive unit 43 and the calculation device 60.

The operation input unit 51 includes an input device such as a joystick, an operation console including various buttons and switches, and a keyboard, and inputs, to the control signal generation unit 52, a signal corresponding to an operation performed from the outside. Specifically, the operation input unit 51 inputs, to the control signal generation unit 52, an operation signal for changing at least one of the position and the posture of the capsule endoscope 10 introduced into the subject 20 depending on an operation performed by the user.

The control signal generation unit 52 generates a control signal for controlling the magnet drive unit 43 of the guidance magnetic field generating device 40 depending on the operation signal input from the operation input unit 51.

The control signal output unit 53 outputs this control signal to the guidance magnetic field generating device 40 and to the calculation device 60.

When guiding the capsule endoscope 10, the magnet drive unit 43 is operated under the control of the guidance magnetic field control device 50, and thereby the external permanent magnet 41 is translated via the support member 42 in each of the horizontal plane and the vertical direction, and the elevation angle and the traverse angle are changed. The position and the posture of the capsule endoscope 10 change following the movement of the external permanent magnet 41.

The calculation device 60 executes a calculation process for calculating the position and the posture of the capsule endoscope 10 based on detection signals of the position-detecting magnetic field output from the signal processor 32 and a calculation process for generating an image of the inside of the subject 20 based on the received signal received by the receiving device 70. The calculation device 60 includes, as illustrated in FIG. 3, a position calculator 601, a correction value acquisition unit 602, a position correction unit 603, a storage unit 604, an image processor 605, and an output unit 606. The position calculator 601 calculates at least one of the position and the posture of the capsule endoscope 10 based on the position-detecting magnetic field generated by the capsule endoscope 10. The correction value acquisition unit 602 acquires correction values for correcting at least one of the position and the posture of the capsule endoscope 10. The position correction unit 603 corrects at least one of the position and the posture of the capsule endoscope 10 calculated by the position calculator 601. The storage unit 604 stores various types of information used in the position detection system 1. The image processor 605 performs a predetermined image process to the received signal received by the receiving device 70, thereby generating image data of an image of the inside of the subject 20 captured by the capsule endoscope 10. The output unit 606 outputs the image of the inside of the subject 20 and various types of information such as the position and the posture of the capsule endoscope 10 to the display device 80.

The position calculator 601 acquires each of the detection signals of the position-detecting magnetic field generated by the capsule endoscope 10 from a plurality of channels (Ch₁ to Ch₁₂ in FIG. 3) of the signal processor 32, and calculates the position and the posture of the capsule endoscope 10 based on these detection signals.

The correction value acquisition unit 602 acquires position information of the capsule endoscope 10 calculated immediately beforehand by the position correction unit 603 from the storage unit 604, acquires a control signal for the guidance magnetic field generating device 40 from the guidance magnetic field control device 50, and acquires, based on the position information and the control signal, correction values for correcting at least one of the position and the posture of the capsule endoscope 10 calculated by the position calculator 601 from a lookup table (LUT) described later.

The position correction unit 603 corrects the position and the posture of the capsule endoscope 10 calculated by the position calculator 601 by using the correction values acquired by the correction value acquisition unit 602, thereby calculating at least one of the corrected position and posture of the capsule endoscope 10.

The storage unit 604 includes a position information storage unit 607, a LUT storage unit 608, and an image data storage unit 609. The position information storage unit 607 stores information indicating the corrected position and posture of the capsule endoscope 10 calculated by the position correction unit 603. The LUT storage unit 608 stores a lookup table (LUT) having information regarding correction values for correcting the position and the posture of the capsule endoscope 10 stored therein. The image data storage unit 609 stores image data of an image generated by the image processor 605. Hereinafter, information indicating the position and the posture of the capsule endoscope 10 is also referred to as position information.

The LUT storage unit 608 stores a lookup table in which at least one of the position and the posture of the capsule endoscope 10, at least one of the position and the posture of the generation source of the interference magnetic field, as well as correction values for at least one of the position and the posture of the capsule endoscope 10 are associated with one another. The correction values here correspond to error in the position and the posture of the capsule endoscope 10 generated depending on a relationship between the relative position and posture of the capsule endoscope 10 and those of the generation source of the interference magnetic field. This lookup table is generated by actually measuring in advance or measuring through a simulation position detection results of the capsule endoscope 10 when the position and the posture of the capsule endoscope 10 and the position and the posture of the generation source of the interference magnetic field are changed, and is stored in the LUT storage unit 608.

The storage unit 604 is achieved by using a ROM, a RAM, or the like. The storage unit 604 stores various control programs and various parameters for controlling each unit of the calculation device 60, a position detection calculation program for the capsule endoscope 10, an image processing program, and the like.

The calculation device 60 having the above configuration is configured, for example, by a computer such as a personal computer or a workstation including a general-purpose processor such as a CPU, a ROM, and a RAM.

The receiving device 70 selects, from a plurality of receiving antennas 71 to be affixed to the body surface of the subject 20 when the examination is performed by the capsule endoscope 10, a receiving antenna 71 having the highest received strength with respect to a radio signal transmitted from the capsule endoscope 10, and performs a demodulation process or the like to the radio signal received via the selected receiving antenna 71, thereby acquiring an image signal and related information.

The display device 80 includes various types of display such as a liquid crystal display and an organic EL display and displays information of an in-vivo image of the subject 20 and the position and the posture of the capsule endoscope 10 on a screen thereof based on the position information and the image data generated in the calculation device 60.

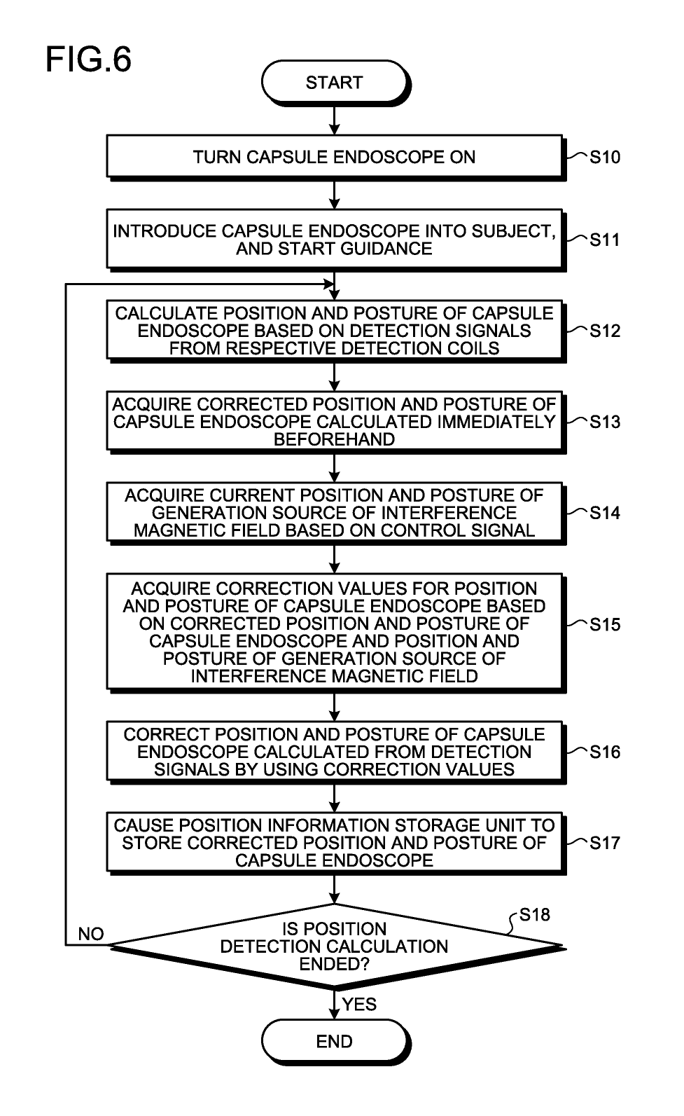

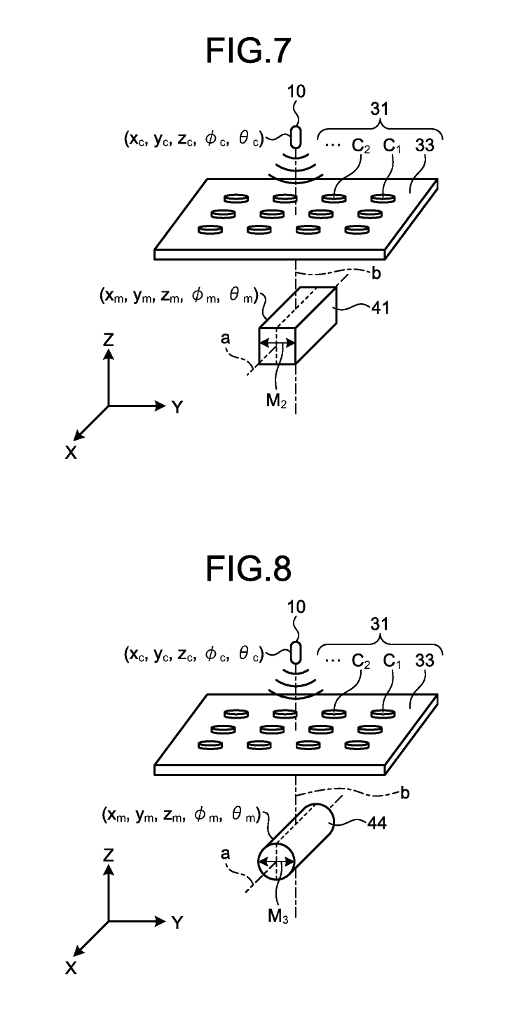

Next, a position detection method according to the first embodiment will be described. FIG. 6 is a flowchart illustrating the position detection method performed by the position detection system 1. FIG. 7 is a schematic view illustrating a positional relationship among the capsule endoscope 10 illustrated in FIG. 3, the plurality of detection coils C₁ to C₁₂, and the external permanent magnet 41. An arrow M₂ in FIG. 7 indicates a magnetization direction of the external permanent magnet 41.

In the following description, in order to simplify the description, it is assumed that only the external permanent magnet 41 is the generation source of the interference magnetic field with respect to the position-detecting magnetic field generated by the capsule endoscope 10, and that the influence of the support member 42 and the magnet drive unit 43 is negligible. In the position correction method described below, it is assumed that a process for correcting a position and a posture of the capsule endoscope 10 is performed.

First, in Step S10, the capsule endoscope 10 is turned on. As a result, power supply from the power supply unit 15 (see FIG. 2) to each unit of the capsule endoscope 10 is started, the imaging unit 11 starts imaging, and the magnetic field generation unit 14 starts generating the position-detecting magnetic field.

In the subsequent Step S11, the capsule endoscope 10 is introduced into the subject 20, and guidance to the capsule endoscope 10 is started. In detail, when the user operates the operation input unit 51 (see FIG. 3), the operation input unit 51 inputs an operation signal corresponding to the input operation to the control signal generation unit 52. In response to the operation signal, the control signal generation unit 52 generates a control signal for changing the position (x, y, z) and the posture (an elevation angle φ, a traverse angle θ) in the three-dimensional space of the external permanent magnet 41. The control signal output unit 53 outputs the control signal to the magnet drive unit 43 and to the correction value acquisition unit 602 of the calculation device 60.

In the subsequent Step S12, the position calculator 601 calculates the position and the posture of the capsule endoscope 10 based on multiple detection signals respectively output from multiple detection coils Cₙ. Specifically, five values (xₛ(tᵢ), yₛ(tᵢ), zₛ(tᵢ), φₛ(tᵢ), θₛ(tᵢ)) indicating the position and the posture of a capsule endoscope 10 at time tᵢ are calculated. Here, the suffix i at the time tᵢ represents the order of time of detection of the position-detecting magnetic field, and i=0, 1, 2, . . . .

In the subsequent Step S13, the correction value acquisition unit 602 acquires from the position information storage unit 607 latest corrected position and posture of the capsule endoscope 10 calculated immediately beforehand by the position correction unit 603. That is, the latest position and posture stored in the position information storage unit 607 are acquired. Specifically, five values (x_{c}(tᵢ₋₁), y_{c}(tᵢ₋₁), z_{c}(tᵢ₋₁), φ_{c}(tᵢ₋₁), θ_{c}(tᵢ₋₁)) indicating the corrected position and posture of the capsule endoscope 10 at time are calculated. When the corrected position and posture of the capsule endoscope 10 have not yet been calculated (that is, when i=0), the correction value acquisition unit 602 may acquire, as data corresponding to the latest corrected position and posture of the capsule endoscope 10, the position and the posture before correction calculated in Step S12, or may acquire preset initial values from the storage unit 604.

In the subsequent Step S14, based on the control signal output from the control signal output unit 53, the correction value acquisition unit 602 acquires current position and posture of the generation source of the interference magnetic field with respect to the position-detecting magnetic field generated by the capsule endoscope 10. Specifically, five values (xₘ(tᵢ), yₘ(tᵢ), zₘ(tᵢ), φₘ(tᵢ), θₘ(tᵢ)) indicating the position and the posture of the external permanent magnet 41 at the time tᵢ are acquired.

In the subsequent Step S15, the correction value acquisition unit 602 acquires correction values for the position and the posture of the capsule endoscope 10 based on the corrected position and posture of the capsule endoscope 10 acquired in Step S13 and the position and the posture of the generation source of the interference magnetic field acquired in Step S14.

In detail, the correction value acquisition unit 602 extracts correction values (Δx, Δy, Δz, Δφ, Δθ) from the lookup table stored in the LUT storage unit 608 employing, as input values, the position and the posture (x_{c}(tᵢ₋₁), y_{c}(tᵢ₋₁), z_{c}(tᵢ₋₁), φ_{c}(tᵢ₋₁), θ_{c}(tᵢ₋₁)) of the capsule endoscope 10 and the position and the posture (xₘ(tᵢ), yₘ(tᵢ), zₘ(tᵢ), φₘ(tᵢ), θₘ(tᵢ)) of the external permanent magnet 41. When the calculation device 60 corrects either one of the position and the posture of the capsule endoscope 10, the correction value acquisition unit 602 extracts only the correction values for the one to be corrected.

In the subsequent Step S16, the position correction unit 603 corrects the position and the posture of the capsule endoscope 10 calculated from the detection signals in Step S12 by using the correction values acquired in Step S15. That is, as expressed by the following formula (1), by respectively subtracting the correction values (Δx, Δy, Δz, Δφ, Δθ) from values (xₛ (tᵢ), yₛ(tᵢ), zₛ(tᵢ), φₛ (tᵢ), θₛ(tᵢ)) indicating the position and the posture of the capsule endoscope 10 calculated from the detection signals, the corrected position and posture (x_{c}(tᵢ), y_{c}(tᵢ), z_{c}(tᵢ), φ_{c}(tᵢ), θ_{c}(tᵢ)) of the capsule endoscope 10 at the time tᵢ are calculated.

$$( \begin{matrix}{x}_{c} ( {t}_{i} ) \\ {y}_{c} ( {t}_{i} ) \\ {z}_{c} ( {t}_{i} ) \\ {ϕ}_{c} ( {t}_{i} ) \\ {θ}_{c} ( {t}_{i} )\end{matrix} ) = ( \begin{matrix}{x}_{s} ( {t}_{i} ) \\ {y}_{s} ( {t}_{i} ) \\ {z}_{s} ( {t}_{i} ) \\ {ϕ}_{s} ( {t}_{i} ) \\ {θ}_{s} ( {t}_{i} )\end{matrix} ) - ( \begin{matrix}Δ x \\ Δ y \\ Δ z \\ Δ ϕ \\ Δ θ\end{matrix} ) \qquad ( 1 )$$

In the subsequent Step S17, the position correction unit 603 causes the position information storage unit 607 to store the corrected position and posture of the capsule endoscope 10.

In the subsequent Step S18, the calculation device 60 determines whether to end a position detection calculation for the capsule endoscope 10. Specifically, the calculation device 60 determines to end the position detection calculation when transmission of the wireless signal from the capsule endoscope 10 has been stopped, a case where a predetermined period of time has passed since the capsule endoscope 10 was turned on, or a case where an operation to end the operation of the calculation device 60 has been performed.

When the position detection calculation is not ended (Step S18: No), the process moves to Step S12. On the other hand, when the position detection calculation is ended (Step S18: Yes), the process is ended.

As described above, according to the first embodiment of the present disclosure, by forming, at least partially, the guidance magnetic field generating device 40 with a conductor, this conductor may be used as a generation source of a known interference magnetic field with respect to the position-detecting magnetic field. Therefore, even when the position and the posture of the generation source of the interference magnetic field change with time, by removing the influence of the interference magnetic field generated by the conductor included in the guidance magnetic field generating device 40 through calculation based on the position and the posture of the generation source and the position and the posture of the capsule endoscope 10, accuracy of detecting the position and the posture of the capsule endoscope 10 may be improved.

Modification

Next, a modification of the first embodiment of the present disclosure will be described. In the first embodiment described above, the correction value acquisition unit 602 acquires the correction values with reference to the lookup table stored in the LUT storage unit 608, but the correction values may be calculated by using functions produced in advance.

In detail, by employing a position and a posture of the capsule endoscope 10 as well as a position and a posture of the generation source of the interference magnetic field (external permanent magnet 41 and the like) as variables (input values), functions which give correction values to the position and the posture of the capsule endoscope 10 are produced in advance and stored in the storage unit 604. As expressed by the following formula (2), the correction values (Δx, Δy, Δz, Δφ, Δθ) are respectively given by functions (fₓ, f_{y}, f_{z}, f_{φ}, f_{θ}) employing, as variables, coordinates (x_{c}, y_{c}, z_{c}), an elevation angle φ_{c}, and a traverse angle θ_{c} of the capsule endoscope 10, as well as coordinates (xₘ, yₘ, zₘ), an elevation angle φₘ, and a traverse angle θₘ of the generation source of the interference magnetic field.

$$( \begin{matrix}Δ x \\ Δ y \\ Δ z \\ Δ ϕ \\ Δ θ\end{matrix} ) = ( \begin{matrix}{f}_{x} ( {x}_{c} , {y}_{c} , {z}_{c} , {ϕ}_{c} , {θ}_{c} , {x}_{m} , {y}_{m} , {z}_{m} , {ϕ}_{m} , {θ}_{m} ) \\ {f}_{y} ( {x}_{c} , {y}_{c} , {z}_{c} , {ϕ}_{c} , {θ}_{c} , {x}_{m} , {y}_{m} , {z}_{m} , {ϕ}_{m} , {θ}_{m} ) \\ {f}_{z} ( {x}_{c} , {y}_{c} , {z}_{c} , {ϕ}_{c} , {θ}_{c} , {x}_{m} , {y}_{m} , {z}_{m} , {ϕ}_{m} , {θ}_{m} ) \\ {f}_{ϕ} ( {x}_{c} , {y}_{c} , {z}_{c} , {ϕ}_{c} , {θ}_{c} , {x}_{m} , {y}_{m} , {z}_{m} , {ϕ}_{m} , {θ}_{m} ) \\ {f}_{θ} ( {x}_{c} , {y}_{c} , {z}_{c} , {ϕ}_{c} , {θ}_{c} , {x}_{m} , {y}_{m} , {z}_{m} , {ϕ}_{m} , {θ}_{m} )\end{matrix} ) \qquad ( 2 )$$

In that case, in Step S15 of FIG. 6, the correction value acquisition unit 602 assigns the corrected position and posture of the capsule endoscope 10 acquired in Step S13 and the position and the posture of the generation source of the interference magnetic field acquired in Step S14 to the above functions, thereby calculating and outputting the correction values of the position and the posture of the capsule endoscope 10.

### Second Embodiment

Next, a second embodiment of the present disclosure will be described. FIG. 7 is a schematic view illustrating an example of a positional relationship among an external permanent magnet, a capsule endoscope, and a plurality of detection coils.

In the second embodiment, the number of input values employed when acquiring correction values is reduced in comparison with that in the first embodiment by using a relative positional relationship between a capsule endoscope 10 and a generation source of an interference magnetic field and symmetry of a shape of the generation source of the interference magnetic field.

A specific description will be given below. For example, when the capsule endoscope 10 is floating in liquid inside a subject 20 (see FIG. 1), as illustrated in FIG. 7, the capsule endoscope 10 is usually constrained by a guidance magnetic field vertically above an external permanent magnet 41, and moves following translational motion in a horizontal plane of the external permanent magnet 41. That is, the coordinates (x_{c}, y_{c}) in the horizontal plane of the capsule endoscope 10 become substantially equal to the coordinates (xₘ, yₘ) in the horizontal plane of the external permanent magnet 41, and in the horizontal plane, error in the position due to the influence of the interference magnetic field hardly occurs. Therefore, in that case, the coordinates (x_{c}, y_{c}) of the capsule endoscope 10 and the coordinates (xₘ, yₘ) of the external permanent magnet 41 may be excluded from the input values employed when the correction value acquisition unit 602 acquires the correction values. In other words, the coordinates (x_{c}, y_{c}) of the capsule endoscope 10 and the coordinates (xₘ, yₘ) of the external permanent magnet 41 may be excluded from the lookup table or the input values in the functions for acquiring the correction values by the correction value acquisition unit 602.

Furthermore, in that case, the capsule endoscope 10 rotates following the rotation of the external permanent magnet 41 about the vertical axis b. That is, the traverse angle θ_{c} of the capsule endoscope 10 becomes substantially equal to the traverse angle θₘ of the external permanent magnet 41, and error in the traverse angle direction due to the influence of the interference magnetic field hardly occurs. Therefore, the traverse angle θ_{c} of the capsule endoscope 10 and the traverse angle θₘ of the external permanent magnet 41 may also be excluded from the input values employed when the correction value acquisition unit 602 acquires the correction values. In other words, the traverse angle θ_{c} of the capsule endoscope 10 and the traverse angle θₘ of the external permanent magnet 41 may also be excluded from the lookup table or the input values in the functions for acquiring the correction values by the correction value acquisition unit 602.

Therefore, in that case, as expressed by the following formulas (3a) to (3e), the correction value acquisition unit 602 acquires the correction values of the position and the posture of the capsule endoscope 10 by employing, as input values, the coordinates z_{c}, zₘ in a vertical direction of the capsule endoscope 10 and the external permanent magnet 41, as well as the elevation angles φ_{c}, φₘ of the capsule endoscope 10 and the external permanent magnet 41 only.

Δx=fₓ(z_{c},φ_{c},zₘ,φₘ) (3a)

Δy=f_{y}(z_{c},φ_{c},zₘ,φₘ) (3b)

Δz=f_{z}(z_{c},φ_{c},zₘ,φₘ) (3c)

Δφ=f_{φ}(z_{c},φ_{c},zₘ,φₘ) (3d)

Δθ=f_{θ}(z_{c},φ_{c},zₘ,φₘ) (3e)

When the calculation device 60 corrects either one of the position and the posture of the capsule endoscope 10, the correction value acquisition unit 602 extracts only the correction values for the one to be corrected.

As described above, according to the second embodiment of the present disclosure, in addition to a similar effect to that in the first embodiment, it is possible to reduce the number of input values used when acquiring the correction values and to reduce a calculation load by using the relative positional relationship between the capsule endoscope 10 and the generation source of the interference magnetic field, and the symmetry of the shape of the generation source of the interference magnetic field.

Modification

Next, a modification of the second embodiment of the present disclosure will be described. A case is considered where, as illustrated in FIG. 8, an external permanent magnet 44 is used which has a rotationally symmetric shape about an axis orthogonal to a magnetization direction. In FIG. 8, the external permanent magnet 44 has a columnar shape. An arrow M₃ in FIG. 8 indicates the magnetization direction of the external permanent magnet 44. In that case, due to the symmetry of the shape of the external permanent magnet 44, the influence of the interference magnetic field on a position-detecting magnetic field does not change even if the external permanent magnet 44 is rotated about the central axis a of rotational symmetry thereof. Therefore, the elevation angle φ_{c} of the capsule endoscope 10 and the elevation angle φₘ of the external permanent magnet 44 may be excluded from the input values employed when the correction value acquisition unit 602 acquires the correction values. In other words, the elevation angle φ_{c} of the capsule endoscope 10 and the elevation angle φₘ of the external permanent magnet 44 may also be excluded from the lookup table or the input values in the functions for acquiring the correction values by the correction value acquisition unit 602.

Therefore, the correction value Δz of the position of the capsule endoscope 10 may be acquired as a correction value only with the coordinates z_{c}, zₘ in the vertical direction of the capsule endoscope 10 and the generation source of the interference magnetic field. Furthermore, when the capsule endoscope 10 is floating in liquid in the subject 20 (see FIG. 1), the coordinate z_{c} in the vertical direction of the capsule endoscope 10 is determined by gravity, buoyancy, and a magnetic attracting force depending on the distance from the external permanent magnet 44, which act on the capsule endoscope 10. Therefore, in that case, as expressed by the following formulas (4a) to (4e), the correction value acquisition unit 602 acquires the correction values of the position and the posture of the capsule endoscope 10 by employing only the coordinate zₘ in the vertical direction of the external permanent magnet 44 as an input value.

Δx=fₓ(zₘ) (4a)

Δy=f_{y}(zₘ) (4b)

Δz=f_{z}(zₘ) (4c)

Δφ=f_{φ}(zₘ) (4d)

Δθ=f_{θ}(zₘ) (4e)

As an example, the following formula (5) indicates a correction formula used when the correction value acquisition unit 602 corrects the position of the capsule endoscope 10 when the coordinate zₘ in the vertical direction of the external permanent magnet 44 as the generation source of the interference magnetic field is employed as an input value.

$$( \begin{matrix}{x}_{c} \\ {y}_{c} \\ {z}_{c}\end{matrix} ) = ( \begin{matrix}{x}_{s} \\ {y}_{s} \\ {z}_{s}\end{matrix} ) - ( \begin{matrix}{k}_{x 6} & {k}_{x 5 } & {k}_{x 4} & {k}_{x 3} & {k}_{x 2} & {k}_{x 1} & {k}_{x 0} \\ {k}_{y 6} & {k}_{y 5} & {k}_{y 4} & {k}_{y 3} & {k}_{y 2} & {k}_{y 1} & {k}_{y 0} \\ {k}_{z 6} & {k}_{z 5} & {k}_{z 4} & {k}_{z 3} & {k}_{z 2} & {k}_{z 1} & {k}_{z 0}\end{matrix} ) ( \begin{matrix}{z}_{m}^{6} \\ {z}_{m}^{5} \\ {z}_{m}^{4} \\ {z}_{m}^{3} \\ {z}_{m}^{2} \\ {z}_{m}^{1} \\ 1\end{matrix} ) \qquad ( 5 )$$

The left-hand side of the formula (5) indicates the position in the three-dimensional space of the capsule endoscope 10 after the correction. The first term on the right-hand side of the formula (5) indicates the position in the three-dimensional space of the capsule endoscope 10 before the correction, that is, the position calculated from the detection signals output from the plurality of detection coils Cₙ. The second term on the right-hand side of the formula (5) indicates correction values (Δx, Δy, Δz) employing the coordinate zₘ in the vertical direction of the external permanent magnet 44 as an input value (variable).

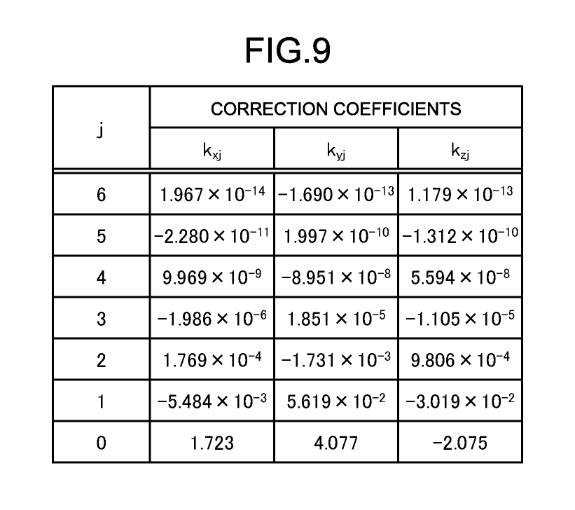

Of the second term on the right-hand side of the formula (5), a matrix of three rows and seven columns is a matrix indicating correction coefficients. Values of respective elements kₓⱼ, k_{yj}, k_{zj} (j=0, 1, 2, 3, 4, 5, 6) of this matrix indicating the correction coefficients are illustrated in FIG. 9. Each value illustrated in FIG. 9 was obtained by a simulation. Of the second term on the right-hand side of the formula (5), the column vector of seven rows and one column is a base vector of a seven-dimensional space configured by using the coordinate zₘ. The correction value acquisition unit 602 calculates the correction values (Δx, Δy, Δz) of the position of the capsule endoscope 10 by performing a calculation with which a matrix indicating the correction coefficients is operated on this column vector.

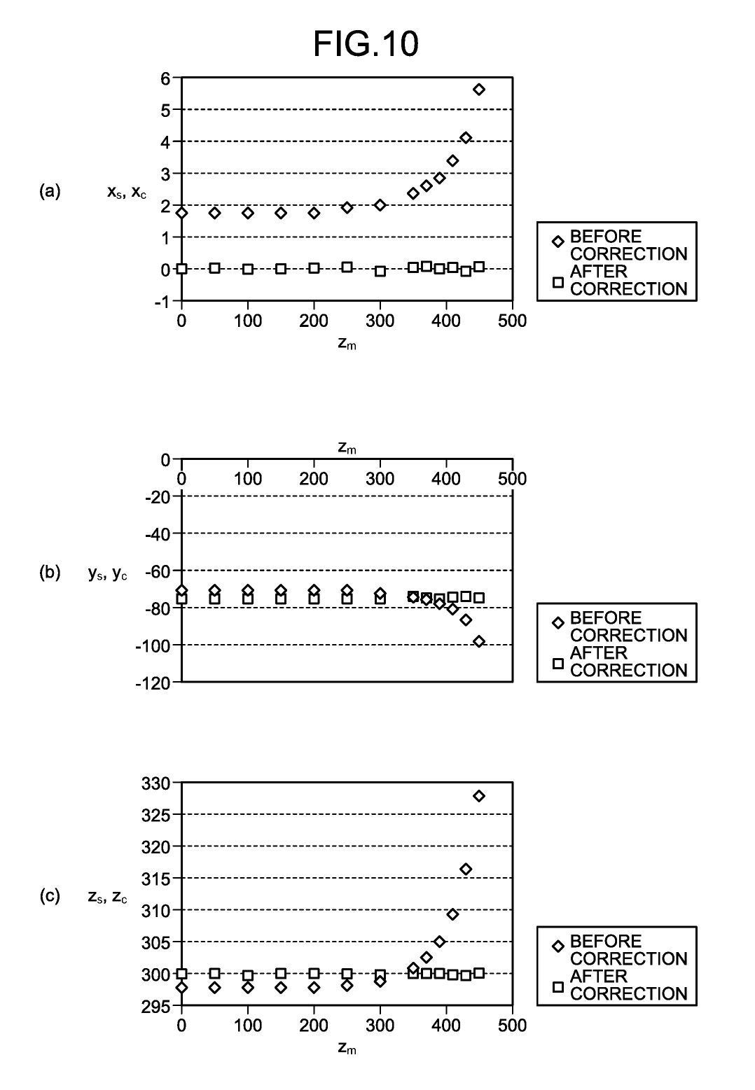

(a) to (c) of FIG. 10 are graphs illustrating a relationship between the coordinate zₘ in the vertical direction of the external permanent magnet 44 and coordinates (xₛ, yₛ, zₛ) of the capsule endoscope 10 before the correction, as well as the coordinate zₘ in the vertical direction of the external permanent magnet 44 and the coordinates (x_{c}, y_{c}, z_{c}) of the capsule endoscope 10 after the correction for each of X-, Y-, and Z-directions. As illustrated in (a) to (c) of FIG. 10, it may be seen that, as the coordinate zₘ in the vertical direction of the external permanent magnet 44 increases, that is, as the external permanent magnet 44 approaches the capsule endoscope 10, the influence of the interference magnetic field increases, and error in the position of the capsule endoscope 10 before the correction increases.

### Third Embodiment

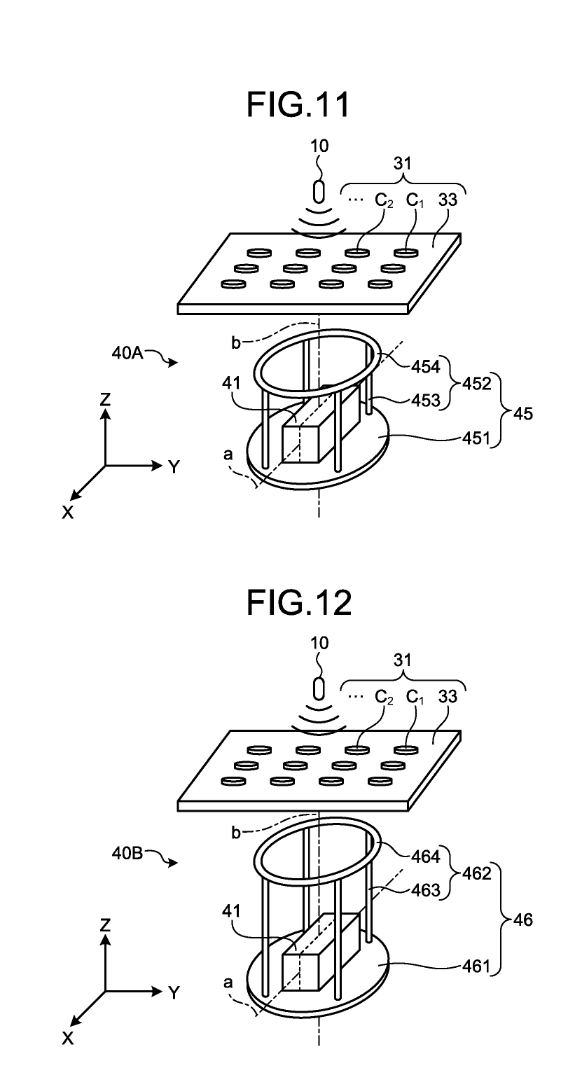

Next, a third embodiment of the present disclosure will be described. FIG. 11 is a schematic view illustrating a partial configuration of a position detection system according to the third embodiment of the present disclosure. The configuration of the position detection system according to the third embodiment is similar to that in the first embodiment as a whole (see FIGS. 1 to 3), and the shape of a support member which supports an external permanent magnet 41 is different from that in the first embodiment.

As illustrated in FIG. 11, a guidance magnetic field generating device 40A according to the third embodiment includes a support member 45 which is capable of translating in a three-dimensional space and supports the external permanent magnet 41 rotatably about a central axis a and a vertical axis b. In FIG. 11, a rotation mechanism which rotates the external permanent magnet 41 in the support member 45 is omitted.

The support member 45 includes a disc-shaped plate material 451 and a frame 452 fixed to the plate material 451. The frame 452 has a plurality of (four in FIG. 11) support columns 453, each of the support columns 453 extending along a vertical direction, and an annular member 454 supported above the plate material 451 by these support columns 453. The whole support member 45 including the plate material 451 and the frame 452 is formed to be rotationally symmetric about a central axis in the vertical direction. In the case illustrated in FIG. 11, this central axis coincides with the vertical axis b.

The plate material 451 and the frame 452 are formed of a conductor such as metal. Therefore, the support member 45 may be a generation source of an interference magnetic field.

Since the frame 452 on an upper surface and side surfaces of the support member 45 does not cover the periphery of the external permanent magnet 41, a guidance magnetic field generated by the external permanent magnet 41 is not shielded by the support member 45, and is generated also in the detection target region R (see FIG. 1). Therefore, by translating the external permanent magnet 41 in the three-dimensional space via the support member 45 and by rotating the external permanent magnet 41 inside the support member 45, the capsule endoscope 10 may be guided by the guidance magnetic field.

The annular member 454 of the frame 452 is arranged so as to be located close to detection coils Cₙ in comparison with the external permanent magnet 41. Accordingly, for the position-detecting magnetic field at the position of the detection coils Cₙ, the influence of the interference magnetic field by the frame 452 is dominant. Therefore, even if the external permanent magnet 41 rotates about the central axis a or the vertical axis b inside the frame 452, the rotation of the external permanent magnet 41 hardly affects detection signals output by the detection coils Cₙ. In that case, it is possible to exclude the elevation angle φₘ and the traverse angle θₘ of the external permanent magnet 41 from the input values employed when the correction value acquisition unit 602 acquires the correction values. In other words, the elevation angle φₘ and the traverse angle θₘ of the external permanent magnet 41 may be excluded from a lookup table or the input values in functions for acquiring the correction values by the correction value acquisition unit 602.

As a result, in a case of using the support member 45, it is possible to reduce the lookup table or the input values in the functions used by the correction value acquisition unit 602 when acquiring the correction values in Step S15 of FIG. 6 to five values (x_{c}, y_{c}, z_{c}, φ_{c}, θ_{c}) which indicate the position and the posture of the capsule endoscope 10, and to three values (xₘ, yₘ, zₘ) which indicate the position of the external permanent magnet 41 (the support member 45).

As described above, according to the third embodiment of the present disclosure, since the support member 45 formed of a conductor serving as a generation source of the interference magnetic field is intentionally disposed as a support member for supporting the external permanent magnet 41, and the external permanent magnet 41 is rotated inside the support member, it is possible to reduce the number of input values used when acquiring the correction values and to reduce a calculation load.

Modification

Next, a modification of the third embodiment of the present disclosure will be described. As described above, when the capsule endoscope 10 is floating in liquid in a subject 20 (see FIG. 1), the capsule endoscope 10 is usually constrained by the guidance magnetic field vertically above the external permanent magnet 41, and moves following translational motion of the external permanent magnet 41 in the horizontal plane. In that case, in the horizontal plane, the coordinates (x_{c}, y_{c}) of the capsule endoscope 10 and the coordinates (xₘ, yₘ) of the external permanent magnet 41 become substantially equal, and error in the position due to the influence of the interference magnetic field hardly occurs. Therefore, in comparison with the third embodiment, the coordinates (xₘ, yₘ) in the horizontal plane of the external permanent magnet 41 (the support member 45) may be further excluded from the input values employed when acquiring the correction values. That is, the input values regarding the external permanent magnet 41 may be reduced to be only the coordinate zₘ in the vertical direction.

### Fourth Embodiment

Next, a fourth embodiment of the present disclosure will be described. FIG. 12 is a schematic view illustrating a partial configuration of a position detection system according to the fourth embodiment of the present disclosure. The configuration of the position detection system according to the fourth embodiment is similar to that in the first embodiment as a whole (see FIGS. 1 to 3), and the shape of a support member which supports an external permanent magnet 41 is different from that in the first embodiment.

As illustrated in FIG. 12, a guidance magnetic field generating device 40B according to the fourth embodiment includes a support member 46 which is capable of translating in a horizontal plane and supports the external permanent magnet 41 rotatably about a central axis a and a vertical axis b and translatably in a vertical direction. In FIG. 12, a rotating mechanism which rotates the external permanent magnet 41 and a moving mechanism which moves the external permanent magnet 41 in the vertical direction in the support member 46 are omitted.

As with the case of the support member 45 illustrated in FIG. 11, the support member 46 includes a disc-shaped plate material 461 and a frame 462 fixed to the plate material 461, and is formed to be rotationally symmetric about a central axis in the vertical direction. In the case illustrated in FIG. 12, this central axis coincides with the vertical axis b. The frame 462 has a plurality of (four in FIG. 12) support columns 463, each of the support columns 463 extending along the vertical direction, and an annular member 464 supported above the plate material 461 by these support columns 463. The length of each of the support columns 463 is longer than that of the support columns 453 illustrated in FIG. 11, and the external permanent magnet 41 may move in the vertical direction within a range of the length of the support columns 463. The annular member 464 is arranged so as to be located close to detection coils Cₙ in comparison with the external permanent magnet 41.

The plate material 461 and the frame 462 are formed of a conductor such as metal. Therefore, the support member 46 may be a generation source of an interference magnetic field.

In the fourth embodiment, the support member 46 is translated only in the horizontal plane while the height in the vertical direction is fixed. Consequently, the annular member 464, which is the generation source of the interference magnetic field which has a dominant influence on a position-detecting magnetic field at the positions of the plurality of detection coils Cₙ, has a constant height. Therefore, even if the external permanent magnet 41 moves in the vertical direction, or rotates about the central axis a or the vertical axis b inside the support member 46, the movement and the rotation of the external permanent magnet 41 hardly affect detection signals output from the plurality of detection coils Cₙ. In that case, the correction value acquisition unit 602 may exclude the coordinate zₘ in the vertical direction, the elevation angle φₘ, and the traverse angle θₘ of the external permanent magnet 41 from the input values employed when acquiring the correction values. In other words, the coordinate zₘ in the vertical direction, the elevation angle φₘ, and the traverse angle θₘ of the external permanent magnet 41 may be excluded from the lookup table or variables in the functions for acquiring the correction values by the correction value acquisition unit 602.

As a result, in a case of using the support member 46, it is possible to reduce the lookup table or the input values in the functions used when acquiring the correction values in Step S15 of FIG. 6 to five values (x_{c}, y_{c}, z_{c}, φ_{c}, θ_{c}) which indicate the position and the posture of the capsule endoscope 10 and to two values (xₘ, yₘ) which indicate the position of the external permanent magnet 41 (the support member 46) in the horizontal plane.

As described above, according to the fourth embodiment of the present disclosure, the support member 46 formed of a conductor serving as a generation source of the interference magnetic field is intentionally disposed as a support member for supporting the external permanent magnet 41, and the external permanent magnet 41 is rotated and moved in the vertical direction inside the support member, and thereby it is possible to further reduce the number of input values used when acquiring the correction values and to reduce a calculation load.

Modification

Next, a modification of the fourth embodiment of the present disclosure will be described. As described above, when the capsule endoscope 10 is floating in liquid in a subject 20 (see FIG. 1), the capsule endoscope 10 is usually constrained by a guidance magnetic field vertically above the external permanent magnet 41, and moves following translational motion of the external permanent magnet 41 in the horizontal plane. Consequently, error in the position due to the influence of the interference magnetic field hardly occurs in the horizontal plane. Therefore, in comparison with the fourth embodiment, the coordinates (xₘ, yₘ) in the horizontal plane of the external permanent magnet 41 (the support member 46) may be further excluded from the input values employed when acquiring the correction values. That is, it is possible to acquire the correction values only with the position and the posture of the capsule endoscope 10.

The first to fourth embodiments of the present disclosure described above and the variations thereof are merely examples for carrying out the present disclosure, and the present disclosure is not limited thereto. In addition, the present disclosure may make various disclosures by appropriately combining a plurality of constituent elements disclosed in the above-mentioned first to fourth embodiments and the variations thereof. It is obvious from the above description that the present disclosure may be variously modified in accordance with specifications and the like, and that various other embodiments are possible within the scope of the present disclosure.

According to the present disclosure, since a guidance magnetic field generating device is at least partially formed of a conductor, and at least one of a position and a posture of a detection target is calculated using at least one of a position and a posture of the conductor, it is possible to accurately detect the position and the posture of the detection target based on a position-detecting magnetic field generated by the detection target even when a position or a posture of a generation source of an interference magnetic field changes.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A position detection system comprising:
a detection target including a magnetic field generator configured to generate an alternating magnetic field for position detection and a permanent magnet provided therein, the detection target being adapted to be introduced into a subject;
a plurality of detection coils arranged outside the subject, each of the detection coils detecting the alternating magnetic field and outputting a detection signal;
a guidance magnetic field generator including
a magnetic field generation source configured to generate a guidance magnetic field for guiding the detection target, and
a driving mechanism configured to change at least one of a position and a posture of the magnetic field generation source,
wherein at least a part of the guidance magnetic field generator is formed of a conductor that generates an interference magnetic field by an action of the alternating magnetic field;
a guidance magnetic field controller configured to control an operation of the driving mechanism; and
a processor comprising hardware, wherein the processor is configured to:
calculate at least one of a position and a posture of the detection target by using: a plurality of the detection signals respectively output from the detection coils; and at least one of a position and a posture of the conductor.

2. The position detection system according to claim 1, wherein the position detector determines a position or a posture of the conductor based on a control signal of the driving mechanism output from the guidance magnetic field controller.

3. The position detection system according to claim 1, wherein
the position detection system includes a storage device configured to store information for associating at least one of the position and the posture of the detection target with a correction value determined in accordance with at least one of: a position or a posture of the detection target; and a position or a posture of the conductor, and
the processor is configured to:
calculate at least one of a position and a posture of the detection target based on the detection signals respectively output by the detection coils;
acquire the correction value from the storage device based on at least one of latest corrected position and posture of the detection target calculated by the position detector and at least one of the position and the posture of the conductor; and
correct at least one of the position and the posture of the detection target calculated by the position calculator by using the correction value.

4. The position detection system according to claim 3, wherein
the storage device stores a lookup table for associating at least one of a position and a posture of the detection target with a correction value determined in accordance with at least one of a position and a posture of the detection target and at least one of a position and a posture of the conductor, and
the processor extracts the correction value from the lookup table by using, as input values, at least one of the latest corrected position and posture of the detection target and at least one of the position and the posture of the conductor.

5. The position detection system according to claim 3, wherein
the storage device stores a function for calculating a correction value for at least one of a position and a posture of the detection target determined in accordance with a relationship between relative positions and postures of the detection target and the conductor by using, as input values, at least one of the position and the posture of the detection target and at least one of the position and the posture of the conductor, and
the processor calculates the correction value by using the function by using, as input values, at least one of the latest corrected position and posture of the detection target and at least one of the position and the posture of the conductor.

6. The position detection system according to claim 3, wherein the magnetic field generation source or the driving mechanism is formed of the conductor.

7. The position detection system according to claim 6, wherein
the magnetic field generation source has a shape that is substantially rotationally symmetric about an axis orthogonal to a magnetization direction, and
the processor acquires the correction value based on positions of the detection target and the magnetic field generation source in a vertical direction.

8. The position detection system according to claim 6, wherein
the magnetic field generation source has a shape that is substantially rotationally symmetric about an axis orthogonal to a magnetization direction, and
the processor acquires the correction value based on a position of the magnetic field generation source in a vertical direction when the detection target is floating in liquid in the subject.

9. The position detection system according to claim 6, wherein
the processor acquires the correction value based on positions of the detection target and the magnetic field generation source in a vertical direction and an elevation angle with respect to a horizontal plane among postures of the detection target and the magnetic field generation source.

10. The position detection system according to claim 6, wherein
the processor acquires the correction value based on the postures of the detection target and the magnetic field generation source.

11. The position detection system according to claim 4, wherein
the guidance magnetic field generator further includes a support that supports the magnetic field generation source,
wherein the support is formed of the conductor,
wherein the support is configured to be rotated by the driving mechanism about two axes orthogonal to each other, and to be translated by the driving mechanism in a three-dimensional space, together with the magnetic field generation source, and
wherein a portion of the support is located closer to the plurality of detection coils than the magnetic field generation source, and
wherein the processor acquires the correction value while excluding the posture of the conductor from the input values.

12. The position detection system according to claim 4, wherein
the guidance magnetic field generator further includes a support that supports the magnetic field generation source,
wherein the support is formed of the conductor,
wherein the support is configured to be rotated by the driving mechanism about two axes orthogonal to each other, to be moved in a vertical direction, and to be translated in a two-dimensional plane, together with the magnetic field generation source, and
wherein a portion of the support is located closer to the plurality of detection coils than the magnetic field generation source, and
wherein the processor acquires the correction value while excluding the posture and the position in the vertical direction of the conductor from the input values.

13. The position detection system according to claim 11, wherein the detection target translates following translational motion in a two-dimensional plane of the conductor, and
the processor acquires the corrected value while excluding a corrected position in a two-dimensional plane of the detection target calculated immediately beforehand by the position detection calculation device and a position in the two-dimensional plane of the conductor from the input values.

14. The position detection system according to claim 1, wherein the detection target is a capsule endoscope including an image sensor configured to generate an image signal by capturing an image of the inside of the subject.

15. An operation method of a position detection system that detects a position of a detection target including a magnetic field generator configured to generate an alternating magnetic field for position detection and a permanent magnet provided therein, the detection target being adapted to be introduced into a subject, wherein the position detection system including:
a plurality of detection coils arranged outside the subject, each of the detection coils detecting the alternating magnetic field and outputting a detection signal;
a guidance magnetic field generator including
a magnetic field generation source configured to generate a guidance magnetic field for guiding the detection target, and
a driving mechanism configured to change at least one of a position and a posture of the magnetic field generation source,
wherein at least a part of the guidance magnetic field generator is formed of a conductor that generates an interference magnetic field by an action of the alternating magnetic field; and
a position detector configured to detect at least one of a position and a posture of the detection target,
the operation method comprising:
calculating, by the position detector, at least one of a position and a posture of the detection target based on the detection signals respectively output by the detection coils;
acquiring, by the position detector, at least one of a position and a posture of the conductor; and
calculating, by the position detector, at least one of a position and a posture of the detection target corrected by using at least one of the position and the posture of the conductor.
